(19) United States
(12) Patent Application Publication
(10) Pub. No.: US 2023/0371930 A1
(43) Pub. Date: Nov. 23, 2023

(54) ULTRASONIC DIAGNOSTIC APPARATUS

(71) Applicant: FUJIFILM Healthcare Corporation, Kashiwa-shi (JP)

(72) Inventors: Akira Kusakabe, Chiba (JP); Koji Waki, Chiba (JP); Eiji Kasahara, Chiba (JP); Suguru Ishiguro, Chiba (JP)

(21) Appl. No.: 18/197,203

(22) Filed: May 15, 2023

(30) Foreign Application Priority Data
May 20, 2022 (JP) 2022-082946

(51) Int. Cl. A61B 8/08; A61B 8/00

(52) U.S. Cl. CPC A61B 8/5207; A61B 8/461; A61B 8/565

(57) ABSTRACT

An information processing unit included in an ultrasonic diagnostic apparatus executes image transfer processing and display processing below. The image transfer processing is processing of successively transferring ultrasonic image data successively generated as time elapses, to an external processing apparatus. The display processing is processing of successively receiving as time elapses externally processed image data transmitted from the external processing apparatus, and causing a display apparatus to display a real-time image in accordance with the externally processed image data. The information processing unit acquires a processing burden value (information indicating a processing ability) of the external processing apparatus from the external processing apparatus, and sets a transfer rate in the image transfer processing in accordance with the processing ability.

### CROSS REFERENCE TO RELATED APPLICATION

This application claims priority to Japanese Patent Application No. 2022-082946 filed on May 20, 2022, which is incorporated herein by reference in its entirety including the specification, claims, drawings, and abstract.

### TECHNICAL FIELD

The present disclosure relates to an ultrasonic diagnostic apparatus, and specifically relates to a technique of transferring data to an external processing apparatus.

### BACKGROUND

Research and development are conducted on a technique of connecting an external processing apparatus to an ultrasonic diagnostic apparatus, and causing the external processing apparatus to execute additional image processing. In this technique, the ultrasonic diagnostic apparatus displays an image based on image data generated by the external processing apparatus. Processing to be executed by the external processing apparatus includes processing of extracting data indicating an outline of a tissue from a series of ultrasonic image data (real-time ultrasonic image data) successively generated as time elapses by the ultrasonic diagnostic apparatus, and drawing the outline of the tissue on a real-time ultrasonic image, and the like.

Patent Documents 1 and 2 below describe techniques of transferring image data generated by an ultrasonic diagnostic apparatus to another apparatus. Moreover, as a technique related to a data transfer technique, Patent Documents 3 to 5 describe techniques of allocating a process to a parallel calculator or parallel calculation processing in accordance with a processing load.

### CITATION LIST

Patent Document 1: JP 2017-47082 A
Patent Document 2: JP 2002-17732 A
Patent Document 3: JP 2004-38715 A
Patent Document 4: JP 2002-49603 A
Patent Document 5: JP 2009-80583 A

### SUMMARY

In the related art, when ultrasonic image data are transferred from an ultrasonic diagnostic apparatus to an external processing apparatus, in a case where the transfer rate of the ultrasonic image data is not suitable to the processing ability of the external processing apparatus, this has imposed a large processing load on the external processing apparatus in some cases.

An object of the disclosure is to obtain a suitable value of the transfer rate when ultrasonic image data are transferred from an ultrasonic diagnostic apparatus to an external processing apparatus.

An ultrasonic diagnostic apparatus according to the disclosure includes an information processing unit that executes: image transfer processing of successively transferring ultrasonic image data successively generated as time elapses, to an external processing apparatus; and display processing of successively receiving as time elapses externally processed image data transmitted from the external processing apparatus, and causing a display apparatus to display a real-time image in accordance with the externally processed image data, in which the information processing unit acquires information indicating a processing ability of the external processing apparatus from the external processing apparatus, and sets a transfer rate in the image transfer processing in accordance with the processing ability.

In one embodiment, the external processing apparatus transmits a variable range of the transfer rate to the information processing unit, and the information processing unit sets the transfer rate in the image transfer processing within the variable range.

In one embodiment, the information processing unit sets a variable range of the transfer rate in accordance with a tissue serving as a diagnosis target, and sets the transfer rate in the image transfer processing within the variable range.

In one embodiment, the information processing unit: sets an operating condition in accordance with a tissue serving as a diagnosis target, and executes preset display processing of causing the display apparatus to display a preset image for setting a variable range of the transfer rate; executes the preset display processing; and sets, in addition to the operating condition, the variable range in accordance with an operation by a user; and sets the transfer rate in the image transfer processing within the variable range.

With the disclosure, a suitable value of the transfer rate when ultrasonic image data are transferred from an ultrasonic diagnostic apparatus to an external processing apparatus is obtained.

### BRIEF DESCRIPTION OF DRAWINGS

Embodiment(s) of the disclosure will be described based on the following figures, wherein:

FIG. 1 is a diagram illustrating a configuration of an ultrasonic diagnostic system;

FIG. 2 is a diagram illustrating configuration examples of an ultrasonic diagnostic apparatus and an external processing apparatus; and

FIG. 3 is a sequence chart of processing that is executed between the ultrasonic diagnostic apparatus and the external processing apparatus.

### DESCRIPTION OF EMBODIMENTS

An embodiment of the disclosure will be described with reference to the drawings. The same components illustrated in a plurality of the drawings are assigned the same reference numerals, and repeated descriptions thereof are omitted.

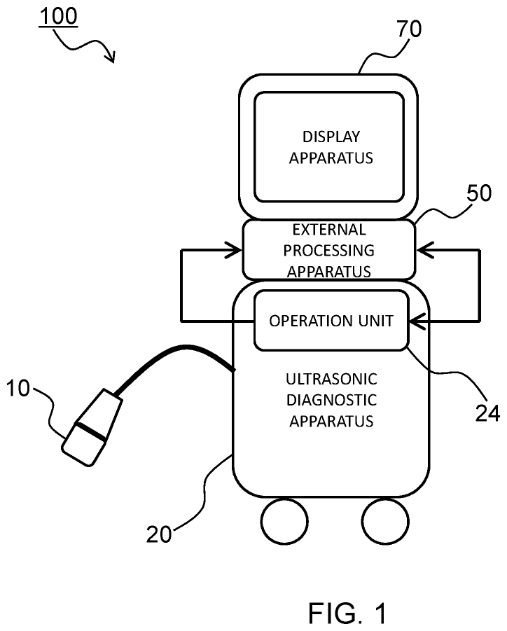

FIG. 1 illustrates an ultrasonic diagnostic system 100 according to the embodiment of the disclosure. The ultrasonic diagnostic system 100 is provided with an ultrasonic diagnostic apparatus 20, an ultrasonic probe 10, an external processing apparatus 50, and a display apparatus 70. When a subject is diagnosed, the ultrasonic probe 10 is in a state where a transmission and reception surface at a tip thereof is in contact with a surface of the subject. The ultrasonic diagnostic apparatus 20 causes the ultrasonic probe 10 to transmit ultrasound. The ultrasound transmitted from the ultrasonic probe 10 is reflected in the subject, and is received by the ultrasonic probe 10. The ultrasonic probe 10 converts the received ultrasound into a reception signal that is an electric signal, and outputs the reception signal to the ultrasonic diagnostic apparatus 20.

The ultrasonic diagnostic apparatus 20 generates image data based on the reception signal. The ultrasonic diagnostic apparatus 20 causes the external processing apparatus 50 to execute additional image processing, and causes the display apparatus 70 to display image data generated by the external processing apparatus 50 thereon. The ultrasonic diagnostic apparatus 20 may cause the display apparatus 70 to display an image based on image data that are not subjected to the processing by the external processing apparatus 50.

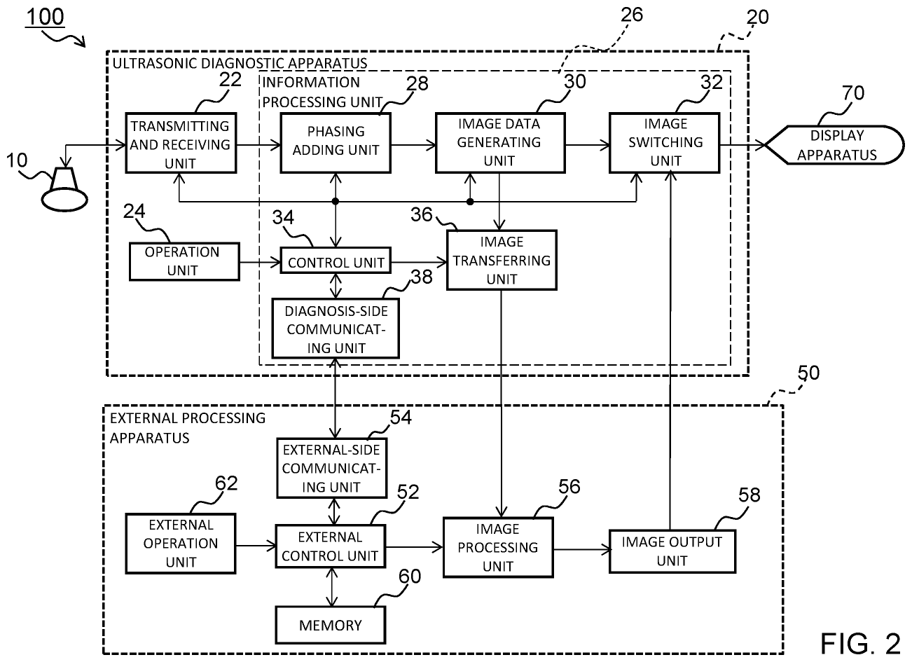

FIG. 2 illustrates configuration examples of the ultrasonic diagnostic apparatus 20 and the external processing apparatus 50. The ultrasonic diagnostic apparatus 20 is provided with a transmitting and receiving unit 22, an operation unit 24, and an information processing unit 26. The information processing unit 26 is provided with a phasing adding unit 28, an image data generating unit 30, an image switching unit 32, a control unit 34, an image transferring unit 36, and a diagnosis-side communicating unit 38. The information processing unit 26 may include processors and electronic circuits that execute programs to implement functions of the respective components (the phasing adding unit 28, the image data generating unit 30, the image switching unit 32, the control unit 34, the image transferring unit 36, and the diagnosis-side communicating unit 38). The image switching unit 32, the image transferring unit 36, and the diagnosis-side communicating unit 38 may include electronic circuits that implement the communication function. The image switching unit 32, the image transferring unit 36, and the diagnosis-side communicating unit 38 may conduct wired communication or wireless communication.

The control unit 34 executes overall control of the ultrasonic diagnostic apparatus 20. The operation unit 24 may be provided with a button, a lever, a keyboard, a mouse, and the like. The operation unit 24 may be a touch panel that is provided to the display apparatus 70. The control unit 34 may execute the control of the ultrasonic diagnostic apparatus 20 based on an operation by a user. The control unit 34 transmits and receives information related to operating conditions of the ultrasonic diagnostic apparatus 20 and the external processing apparatus 50, to and from the external processing apparatus 50 via the diagnosis-side communicating unit 38.

The ultrasonic probe 10 is provided with a plurality of ultrasonic transducers. The transmitting and receiving unit 22 outputs transmission signals that are electric signals to the plurality of the ultrasonic transducers. Each of the ultrasonic transducers converts the transmission signal to ultrasound, and transmits the ultrasound toward a subject. The transmitting and receiving unit 22 adjusts delay time of the transmission signal to be output to each of the ultrasonic transducers to form an ultrasonic beam in a specific direction.

Each of the ultrasonic transducers receives ultrasound reflected in the subject, converts the ultrasound to a reception signal that is an electric signal, and outputs the reception signal to the phasing adding unit 28. The phasing adding unit 28 adjusts the delay time of the reception signal output from each of the ultrasonic transducers such that reception signals by the ultrasound arrived from the direction toward which the ultrasonic beams are directed are caused to constructively interfere with each other, and adds and sums the respective reception signals after the delay time adjustment. The phasing adding unit 28 outputs a phasing addition signal generated in this manner to the image data generating unit 30.

Further, the transmitting and receiving unit 22 changes the delay time of a transmission signal to be output to each of the ultrasonic transducers such that ultrasonic beams are scanned within a specific observation cross-section in the subject. Moreover, the phasing adding unit 28 changes the delay time of a reception signal output from each of the ultrasonic transducers such that a phasing addition signal corresponding to the direction of the ultrasonic beam scanned in the subject is generated, and adds and sums the respective reception signals after the delay time adjustment.

The image data generating unit 30 generates B mode image data based on the phasing addition signals acquired in the respective directions within the observation cross-section, and outputs the B mode image data to the image switching unit 32. The transmitting and receiving unit 22, the phasing adding unit 28, and the image data generating unit 30 successively generate B mode image data at a predetermined frame rate as time elapses. Here, the frame rate indicates the number of sheets of B mode images to be generated per unit time.

In basic display processing that is executed in the ultrasonic diagnostic system 100, the image switching unit 32 outputs B mode image data successively generated as time elapses to the display apparatus 70. The display apparatus 70 displays an image based on the B mode image data successively generated as time elapses; in other words, a real-time image of the B mode image.

In the ultrasonic diagnostic system 100, application display processing below may be executed. The image data generating unit 30 outputs B mode image data successively generated as time elapses to the image transferring unit 36. The image transferring unit 36 transfers the B mode image data successively generated as time elapses to the external processing apparatus 50 at a transfer rate set by the control unit 34. The transmitting and receiving unit 22, the phasing adding unit 28, and the image data generating unit 30 may successively generate B mode image data as time elapses, at a frame rate in accordance with the transfer rate.

The external processing apparatus 50 is provided with an external control unit 52, an external-side communicating unit 54, an image processing unit 56, an image output unit 58, a memory 60, and an external operation unit 62. The external control unit 52 and the image processing unit 56 may include processors and electronic circuits that execute programs to implement functions of these components (the external control unit 52 and the image processing unit 56). The external-side communicating unit 54, the image processing unit 56, and the image output unit 58 may include electronic circuits that implement the communication function. The external-side communicating unit 54, the image processing unit 56, and the image output unit 58 may conduct wired communication or wireless communication.

The external processing apparatus 50 may be a general computer such as a personal computer, a tablet computer, a workpiece station, or the like. The external control unit 52 performs overall control of the external processing apparatus 50. The external operation unit 62 may be a keyboard, a mouse, and the like. In a case where the external processing apparatus 50 is provided with a display device such as a display, the external operation unit 62 may be a touch panel included in the display device. The external operation unit 62 may be configured integrally with the operation unit 24 of the ultrasonic diagnostic apparatus 20 with common hardware.

The external control unit 52 may control the external processing apparatus 50 in accordance with an operation of the external operation unit 62 by the user. The external-side communicating unit 54 transmits and receives signals to and from the diagnosis-side communicating unit 38. The external control unit 52 transmits and receives information related to operating conditions of the ultrasonic diagnostic apparatus 20 and the external processing apparatus 50, to and from the ultrasonic diagnostic apparatus 20 via the external-side communicating unit 54 and the diagnosis-side communicating unit 38. For example, the external control unit 52 may transmit a command about information to be displayed on the display apparatus 70, in accordance with the operation of the external operation unit 62 by the user, to the control unit 34 of the ultrasonic diagnostic apparatus 20 via the external-side communicating unit 54 and the diagnosis-side communicating unit 38.

The image processing unit 56 receives B mode image data successively transferred as time elapses from the image transferring unit 36 of the ultrasonic diagnostic apparatus 20, as data indicating an ultrasonic image in real time (hereinafter, referred to as a real-time ultrasonic image in some cases). The image processing unit 56 performs additional image processing on the real-time ultrasonic image. The additional image processing includes processing of extracting an outline of a tissue from the real-time ultrasonic image and drawing the outline of the tissue on the real-time ultrasonic image, and the like.

The image processing unit 56 generates outline data indicating an outline of a tissue from the B mode image data successively transferred as time elapses, synthesizes the outline data and the B mode image data, and successively generates outline-B mode image data as time elapses. The outline-B mode image data are data indicating an image in which a figure indicating the outline (figure of the outline drawn by the line) is overlapped with the B mode image.

The additional image processing may be processing (fusion display processing) of arranging side by side a basic image such as a magnetic resonance imaging (MRI) image or a computed tomography (CT) image, and a real-time ultrasonic image, relative to a common observation cross-section in the subject. The image processing unit 56 reads in the basic image stored in the memory 60 in accordance with the control by the external control unit 52. Moreover, the image processing unit 56 may read in a basic image from an external apparatus. In order to execute the fusion display processing, for example, a position sensor is provided to the ultrasonic probe 10. Based on a position of the ultrasonic probe 10 detected by the position sensor, a basic image in a cross-section where a real-time ultrasonic image has been observed is displayed side by side with the real-time ultrasonic image.

The image processing unit 56 synthesizes an image indicating basic image data and B mode image data, and successively generates as time elapses basic-B mode image data indicating the basic image and the B mode image by being arranged side by side.

The image processing unit 56 successively outputs as time elapses externally processed image data such as the outline-B mode image data or the basic-B mode image data, to the image output unit 58. The image output unit 58 successively transmits as time elapses the externally processed image data to the image switching unit 32 of the ultrasonic diagnostic apparatus 20. The image switching unit 32 successively outputs as time elapses the externally processed image data to the display apparatus 70. The display apparatus 70 displays an image based on the externally processed image data successively generated as time elapses; in other words, a real-time ultrasonic image in which figures indicating the outline of the tissue are overlapped, or the basic image and the real-time ultrasonic image by being arranged side by side.

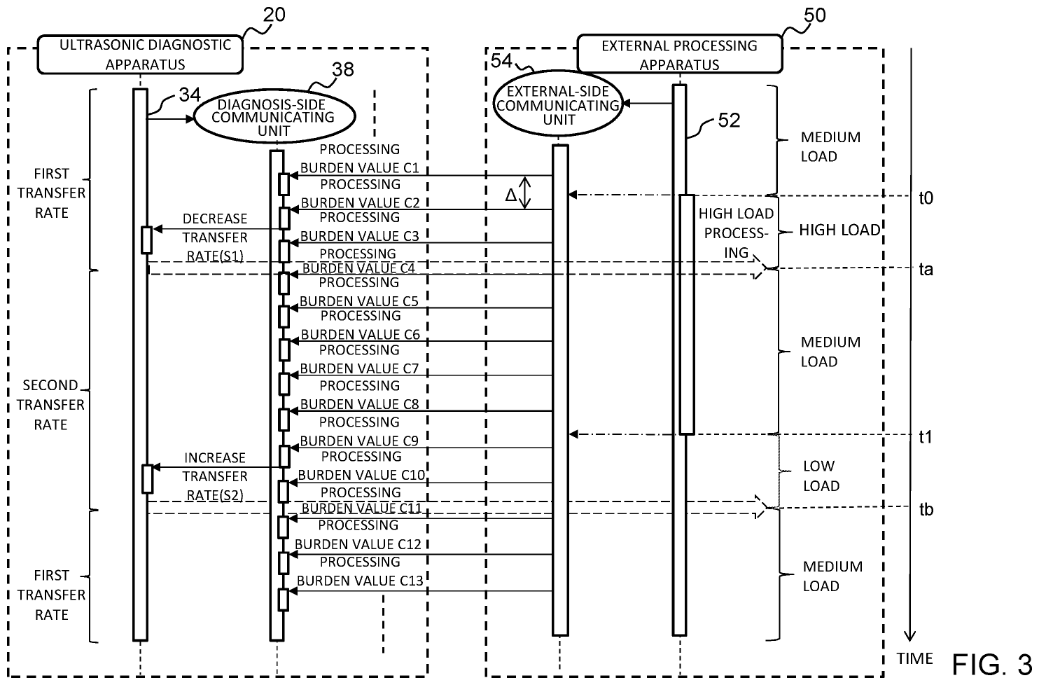

FIG. 3 illustrates a sequence chart of processing that is executed between the ultrasonic diagnostic apparatus 20 and the external processing apparatus 50. The external control unit 52 transmits a processing burden value at a predetermined time interval A via the external-side communicating unit 54 to the ultrasonic diagnostic apparatus 20. The processing burden value may be a value indicating a rate of an amount of information that is processed by the external processing apparatus 50 per unit time at a current time point with respect to an amount of information (bytes) that can be processed by the external processing apparatus 50 per unit time. The processing burden value may be a CPU use rate or a memory use rate.

The control unit 34 of the ultrasonic diagnostic apparatus 20 receives a processing burden value transmitted from the external processing apparatus 50 at the predetermined time interval A via the diagnosis-side communicating unit 38. The control unit 34 determines a transfer rate when the image transferring unit 36 transfers B mode image data to the external processing apparatus 50, in accordance with the processing burden value. In the present embodiment, the control unit 34 sets the transfer rate to a first transfer rate if the processing burden value is less than a predetermined burden threshold, and the control unit 34 sets the transfer rate to a second transfer rate lower than the first transfer rate if the processing burden value is equal to or greater than the burden threshold.

In the example illustrated in FIG. 3, from time t0 to time t1, the external processing apparatus 50 executes high load processing. The high load processing may be, for example, processing of generating externally processed image data. The external control unit 52 transmits processing burden values . . . C1, C2, C3, C13, . . . at the time interval Δ to the ultrasonic diagnostic apparatus 20. The external control unit 52 starts the high load processing at the time t0, and thus transmits, immediately after the time t0, the processing burden value C2 more than the processing burden value C1 transmitted before the time t0, to the ultrasonic diagnostic apparatus 20.

The control unit 34 having received the processing burden value C2 determines a transfer rate in accordance with the processing burden value C2. In the example illustrated in FIG. 3, the processing burden values C2 to C8 that are transmitted from the external processing apparatus 50 to the ultrasonic diagnostic apparatus 20 between the time t0 and the time t1 are equal to or greater than the burden threshold. On the other hand, the processing burden value C1 transmitted from the external processing apparatus 50 to the ultrasonic diagnostic apparatus 20 before the time t0, and processing burden values C9, C10 . . . C13 transmitted from the external processing apparatus 50 to the ultrasonic diagnostic apparatus 20 after the time t1 are less than the burden threshold. In response to the reception of the processing burden value C2, the control unit 34 changes the transfer rate from the first transfer rate to the second transfer rate, and decreases the transfer rate (S1). The external processing apparatus 50 ends the high load processing at the time t1, and thus transmits, immediately after the time t1, the processing burden value C9 less than the processing burden value C8 transmitted to the ultrasonic diagnostic apparatus 20 before the time t1, to the ultrasonic diagnostic apparatus 20.

The processing burden value C9 is equal to or greater than the burden threshold as mentioned above. In response to the reception of the processing burden value C9, the control unit 34 changes the transfer rate from the second transfer rate to the first transfer rate, and increases the transfer rate (S2).

The degree of magnitude of the processing load in the external processing apparatus 50 is illustrated by being classified into three stages of a low load, a medium load, and a high load, at the right side from the external processing apparatus 50 in FIG. 3. The processing load is defined, for example, using an amount of information (bytes) that is processed by the external processing apparatus 50 per unit time. With the start of the high load processing at the time t0, the processing load becomes the high load immediately thereafter, but the processing load becomes the medium load from the high load at time to due to a decrease in the transfer rate (S1).

Thereafter, with the end of the high load processing at the time t1, the processing load becomes the low load, but the processing load becomes the medium load from the low load at time tb due to an increase in the transfer rate (S2).

In this manner, the information processing unit 26 (the phasing adding unit 28, the image data generating unit 30, the image switching unit 32, the control unit 34, the image transferring unit 36, and the diagnosis-side communicating unit 38) included in the ultrasonic diagnostic apparatus 20 according to the present embodiment executes image transfer processing and display processing below. The image transfer processing is processing of successively transferring ultrasonic image data successively generated as time elapses, to the external processing apparatus 50. The display processing is processing of successively receiving as time elapses externally processed image data transmitted from the external processing apparatus 50, and causing the display apparatus 70 to display a real-time image in accordance with the externally processed image data. The information processing unit 26 acquires a processing burden value (information indicating a processing ability) of the external processing apparatus 50 from the external processing apparatus 50, and sets a transfer rate in the image transfer processing in accordance with the processing ability.

In a case where the external processing apparatus 50 executes the high load processing, when B mode image data are transferred from the ultrasonic diagnostic apparatus 20 at the same transfer rate as that when normal processing is executed, the image processing unit 56 has difficulty executing the image processing at a sufficient speed for the B mode image data in some cases. In other words, the image processing unit 56 has difficulty executing image processing that follows the B mode image data in some cases.

Therefore, it can be considered that the quality of each B mode image is lowered by decreasing the number of pixels of the B mode image data, for example, and the high load processing is executed while maintaining the transfer rate. However, in some purposes and the like of diagnoses, lowering the quality of the B mode images is not preferable in some cases.

In the ultrasonic diagnostic system 100 according to the present embodiment, the transfer rate at which B mode image data are transferred from the ultrasonic diagnostic apparatus 20 to the external processing apparatus 50 is determined in accordance with a processing load of the external processing apparatus 50. This makes it easy to cause the processing by the external processing apparatus 50 to follow B mode image data that are transferred from the ultrasonic diagnostic apparatus 20, while maintaining the quality of the B mode image serving as a processing target of the external processing apparatus 50. Moreover, when the processing load of the external processing apparatus 50 is small, a smooth real-time ultrasonic image is displayed due to a high transfer rate.

An application embodiment of the disclosure will be described. In the above, the processing in which the control unit 34 determines the transfer rate as either of the first transfer rate and the second transfer rate in accordance with a processing burden value transmitted from the external processing apparatus 50 has been described. The transfer rate may be adjusted more minutely. For example, the control unit 34 may determine the transfer rate as any of the first transfer rate to an N-th transfer rate in accordance with the processing burden value transmitted from the external processing apparatus 50. Herein, N is an integer of two or more, and the values of the first transfer rate, the second transfer rate, . . . and the N-th transfer rate decrease in this order. The control unit 34 makes the transfer rate higher as the processing burden value is smaller, and makes the transfer rate lower as the processing burden value is larger.

In this case, a variable range may be determined with respect to the transfer rate that is determined by the control unit 34. The variable range is defined by a lower limit value and an upper limit value. The external control unit 52 may read in the variable range by an operation of the external operation unit 62 of the external processing apparatus 50. The external control unit 52 transmits information indicating the variable range to the control unit 34 via the external-side communicating unit 54 and the diagnosis-side communicating unit 38. When the transfer rate determined based on the processing burden value has exceeded the upper limit value of the variable range, the control unit 34 changes the transfer rate to the upper limit value. Moreover, when the transfer rate determined based on the processing burden value has become less than the lower limit value of the variable range, the control unit 34 changes the transfer rate to the lower limit value. When the transfer rate determined based on the processing burden value transmitted from the external processing apparatus 50 is within the variable range, the control unit 34 maintains the value of the transfer rate.

Note that, the upper limit value and the lower limit value may be the same fixed value, and in this case, the transfer rate is the fixed value. The same applies to the upper limit value and the lower limit value of the variable range below.

With such processing, the transfer rate is prevented from extremely lowering, and the smoothness of the motion of a real-time ultrasonic image that is indicated by the externally processed image data is maintained. Moreover, the transfer rate is prevented from rising drastically, and the processing by the external processing apparatus 50 is easily caused to follow the B mode image data that are transferred from the ultrasonic diagnostic apparatus 20. In addition, the quality of the B mode image that is illustrated by the B mode image data is easily improved.

The control unit 34 may set a variable range of the transfer rate in accordance with a tissue serving as a diagnosis target (an organ, a site of the body, or the like) specified in advance. For example, a lower limit value of the variable range of the transfer rate in a case where an organ that does not move actively, such as a liver or a kidney, serves as a diagnosis target, may be set smaller than that in a case where an organ that moves actively, such as a heart, serves as a diagnosis target. Moreover, an upper limit value of the variable range of the transfer rate in a case where an organ that moves actively serves as a diagnosis target may be set larger than that in a case where an organ that does not move actively serves as a diagnosis target.

A diagnosis target may be specified due to an operation by a user with respect to the operation unit 24. In other words, the control unit 34 recognizes the tissue serving as a diagnosis target in accordance with the operation by the user, and sets the variable range of the transfer rate in accordance with the tissue serving as a diagnosis target. Moreover, in a case where the ultrasonic diagnostic apparatus 20 has a preset function for each tissue, a variable range of the transfer rate corresponding to a tissue that is specified as a diagnosis target may be set when the preset function for each tissue is activated. Herein, the preset function indicates a function of setting an operating condition to a tissue serving as a diagnosis target. In the preset function, there is executed processing of causing the display apparatus 70 to display an image for setting each control parameter with respect to each function of the ultrasonic diagnostic apparatus 20, and causing the user to perform setting of each control parameter.

Examples of the control parameters include the depth of the observation range, a gain with respect to the reception signal, time gain control (control parameter for adjusting a state in which the gain is increased as the reception time elapses), the focus (the degree of converging the ultrasonic beam), and the like.

When an operation for activating the preset function is performed in the operation unit 24, the control unit 34 generates preset image data indicating a format with which the respective control parameters are input in the image data generating unit 30, and outputs the preset image data to the image switching unit 32. The image switching unit 32 outputs the preset image data to the display apparatus 70. The display apparatus 70 displays a preset image based on the preset image data. The preset image indicates, in addition to the format with which the respective control parameters are input, a format with which the upper limit value and the lower limit value of the variable range of the transfer rate are input. In response to the operation by the operation unit 24 in a state where the preset image is displayed, the control unit 34 reads in the respective control parameters, and the upper limit value and the lower limit value of the variable range.

In this manner, the information processing unit 26 may set a variable range of the transfer rate in accordance with the tissue serving as a diagnosis target, and set the transfer rate in the image transfer processing within the variable range. Specifically, the information processing unit 26 sets an operating condition in accordance with a tissue serving as a diagnosis target, and executes preset display processing of causing the display apparatus 70 to display a preset image for setting a variable range of the transfer rate. Moreover, the information processing unit 26 executes the preset display processing, sets an operating condition and a variable range in response to an operation by a user, and sets the transfer rate in the image transfer processing within the variable range.

The ultrasonic diagnostic system 100 may be configured so as to be able to select either one of an operation in an adaptive control mode in which the transfer rate is changed in accordance with the processing burden value as mentioned above, and an operation in a fixed mode in which the transfer rate is fixed. The external control unit 52 transmits, in accordance with the operation of the external operation unit 62 by the user, mode setting information for setting an operation mode of the ultrasonic diagnostic apparatus 20, to the control unit 34 via the external-side communicating unit 54 and the diagnosis-side communicating unit 38. The control unit 34 operates in a mode in accordance with the mode setting information.

The external control unit 52 and the image processing unit 56 may adjust the quality of the B mode image serving as a processing target in accordance with the transfer rate that is transmitted from the ultrasonic diagnostic apparatus 20, when operating in the fixed mode. The external control unit 52 and the image processing unit 56 may lower the quality of the B mode image more, for example, as the transfer rate is higher. Therefore, the image processing is executed at a sufficient speed with respect to the B mode image data that is successively transferred as time elapses from the ultrasonic diagnostic apparatus 20. On the other hand, when the transfer rate is low, the quality of the B mode image is maintained, and a real-time image with high resolution is displayed.

In the forgoing, the embodiment in which the ultrasonic diagnostic apparatus 20 and the external processing apparatus 50 are housed in separate housings has been described. The ultrasonic diagnostic apparatus 20 and the external processing apparatus 50 may be housed in the same housing. In this case, the control unit 34 that executes the processing of the ultrasonic diagnostic apparatus 20, and the external control unit 52 that executes the processing of the external processing apparatus 50 may be configured as separate devices (CPUs and the like), and may be housed in the same housing.

Moreover, the external processing apparatus 50 may be connected to the ultrasonic diagnostic apparatus 20 via a communication channel. The communication channel may be a wired communication channel or may be a wireless communication channel. In this case, the external processing apparatus 50 may be disposed in a room different from a room where the ultrasonic diagnostic apparatus 20 is disposed.

In the forgoing, the embodiment in which the image data that are transferred from the ultrasonic diagnostic apparatus 20 to the external processing apparatus 50 are B mode image data has been described. The image data that are transferred from the ultrasonic diagnostic apparatus 20 to the external processing apparatus 50 may be other image data that are obtained by transmission and reception of ultrasounds with respect to a subject. The image data may be, for example, M mode image data indicating a motion of a tissue or Doppler image data indicating the speed of the blood flow.

1. An ultrasonic diagnostic apparatus comprising an information processing unit that executes:
image transfer processing of successively transferring ultrasonic image data successively generated as time elapses, to an external processing apparatus; and
display processing of successively receiving, as time elapses, externally processed image data transmitted from the external processing apparatus, and causing a display apparatus to display a real-time image in accordance with the externally processed image data, wherein
the information processing unit acquires information indicating a processing ability of the external processing apparatus from the external processing apparatus, and sets a transfer rate in the image transfer processing in accordance with the processing ability.

2. The ultrasonic diagnostic apparatus according to claim 1, wherein
the external processing apparatus transmits a variable range of the transfer rate to the information processing unit, and
the information processing unit sets the transfer rate in the image transfer processing within the variable range.

3. The ultrasonic diagnostic apparatus according to claim 1, wherein
the information processing unit sets a variable range of the transfer rate in accordance with a tissue serving as a diagnosis target, and sets the transfer rate in the image transfer processing within the variable range.

4. The ultrasonic diagnostic apparatus according to claim 1, wherein
the information processing unit:
sets an operating condition in accordance with a tissue serving as a diagnosis target, and executes preset display processing of causing the display apparatus to display a preset image for setting a variable range of the transfer rate;
executes the preset display processing, and sets, in addition to the operating condition, the variable range in accordance with an operation by a user; and
sets the transfer rate in the image transfer processing within the variable range.
